(19) United States
(12) Patent Application Publication
(10) Pub. No.: US 2023/0405123 A1
(43) Pub. Date: Dec. 21, 2023

(54) COMPOSITIONS OF NANOPARTICLES FOR TREATMENT OF CANCER

(71) Applicant: NANOBIOTIX S.A., PARIS (FR)

(72) Inventors: ELSA BORGHI, PARIS (FR); KATHERINE JAMESON, CAMBRIDGE, MA (US)

(21) Appl. No.: 18/035,303

(22) PCT Filed: Oct. 22, 2021
(86) PCT No.: PCT/EP2021/079399

§ 371 Date: May 4, 2023

(30) Foreign Application Priority Data
Nov. 5, 2020 (EP) 20306326.8

(51) Int. Cl. A61K 41/00; C07K 16/28; A61P 35/04; A61K 33/24; A61K 39/395; A61K 9/51

(52) U.S. Cl. CPC A61K 41/0038; C07K 16/2818; A61P 35/04; A61K 33/24; A61K 39/3955; A61K 9/5115; C07K 2317/21; C07K 2317/24; A61K 2039/505

(57) ABSTRACT

The invention relates to nanoparticles and/or aggregates of nanoparticles and a composition comprising nanoparticles and/or aggregates of nanoparticles and their use in oncology. Specifically, the nanoparticles and/or aggregates of nanoparticles are radiation enhancer agents to be activated by ionizing radiation, and are for use, in combination with at least one immunooncology (IO) agent, in the treatment of malignant tumors in human patients who have failed to respond to a previous immunotherapy and/or radiotherapy (RT) and who experience disease progression.

### TECHNICAL DOMAIN

The invention concerns nanoparticles and/or aggregates of nanoparticles and a composition comprising nanoparticles and/or aggregates of nanoparticles and their use in oncology.

Specifically, the nanoparticles and/or aggregates of nanoparticles are radiation enhancer agents to be activated by ionizing radiation, and are for use in combination with at least one immunooncology (IO) agent, in the treatment of malignant tumors in human patients who have previously been administered with a treatment involving immunotherapy and/or radiotherapy (RT) for the same disease.

### PREAMBLE

Many options for the treatment of tumorous cancers exist today. Tumor treatment may be local, including surgery (if the tumor is accessible and can be safely isolated in surgery) and radiotherapy (RT), as well as systemic (e.g., administering cytotoxics or molecular targeted therapies).

Immuno-oncology (IO) agents (also referred to as cancer immunotherapeutic agents) harness the body's own immune system to kill cancer cells. For example, immune checkpoint inhibitors (ICIs), in the form of antibodies are currently used in the clinic, like ipilimumab that targets CTLA-4, or ICIs targeting the PD1/PD-L1 axis. Another class of IO agent, chimeric antigen receptor (CAR) T cells, is now approved for certain types of blood cancers.

However, in a recent review of IO therapy [Hegde and Chen “Top 10 Challenges in Cancer Immunotherapy” Immunity, 52 (2020) pp. 17-35], the authors indicate that “only a minority of patients with otherwise terminal cancer experience life-altering durable survival from these [IO] therapies. These outcomes likely reflect the complex and highly regulated nature of the immune system.”. This means that only approximately 15% of patients receiving ICIs actually respond to the treatment, with some initial responders eventually developing resistance [Gong et al. (2018) Development of PD-1 and PD-L1 inhibitors as a form of cancer immunotherapy: a comprehensive review of registration trials and future considerations. J. Immunother. Cancer, 6(1):8].

With respect to radiotherapy, the radiation dose and ultimate efficacy of RT is limited by the potential toxicity to surrounding healthy tissues. Biological methods to optimize the RT efficacy include accelerated fractionation, hyper fractionation, and stereotactic body radiation therapy (SBRT) (also called stereotactic ablative radiotherapy (SABR)). Physical methods to optimize the RT efficacy include delivering a much higher dose of radiation to the tumor than to neighboring healthy tissues and/or organs at risk, for example via targeted image-guided treatment with intensity modulated RT (IMRT). FLASH-RT delivery uses irradiators with a high radiation output that allows for the entire RT treatment, or large fraction doses, to be delivered in parts of a second, for example, 15 Gy in 90 ms, compared to several minutes for convention RT.

Another recent approach to reducing radiation toxicity and improving the benefit/risk ratio of RT involves the administration (e.g., by intra tumoral injection) of “radioenhancer agents” or “radioenhancers”. Their presence in the tumor increases the radiation energy dose deposit within the tumor mass without increasing the radiation energy dose deposit in the surrounding healthy tissues [L. Maggiorella et al. Nanoscale radiotherapy with hafnium oxide nanoparticles. Future Oncol. (2012) 8(9), 1167-118].

Following an anti-cancer treatment, several clinical outcomes are possible, including a complete response (CR), a partial response (PR), or only stable disease (SD) or, in the worst case, progressive disease (PD). In some cases, a patient may experience CR, PR or SD, for months or even years, which is then followed by disease progression. These response criteria are defined according to RECIST 1.1 criteria [European Journal of Cancer 45 (2009) 228-247 “New response evaluation criteria in solid tumors: Revised RECIST guideline (version 1.1)”],

Patients may, for example, after an initial CR after a previous anti-cancer treatment, present a loco-regional tumor and/or other distant metastases. A loco-regional recurrence (LRR) tumor is a (cancerous) tumor that had been fully or partially controlled in a previous therapy, but then regrows at or close to the site of the initial tumor. When the LRR tumor is accompanied by further distant metastases, one refers to an LRR/Met (also herein identified as “LRR/M”) disease state. For example, in head and neck cancer, post-surgery radiation, or (chemoradiation) are well established treatment regimens used to reduce the risk of recurrence, but still, up to 30% of patients recur [https://www.spohnc.org/recurrent-and-metastatic-head-and-neck-cancer/].

In other cases, even if the primary tumor appears to be controlled due to the previously administered treatment, the disease may progress to an oligometastatic state or to a widespread metastatic state. The oligometastatic cancer state or oligometastatic disease has been defined as an intermediate phenotype between locoregionally confined malignancy and widespread metastatic disease, largely characterized by clinical features, including a numerically limited number (1-5) of metastases and a slow pace of progression [Hellman & Weichselbaum (1995) J. Clin Oncol. 13: 8-10].

The treatment options for these LRR, LRR/Met or oligometastatic disease state patients, who have received a previous treatment for the same cancer, are somewhat limited. The re-introduction of the same class of therapeutic agent(s) (e.g., same class of cytoxic agent(s), same class of IO agent(s), and/or RT) used during the previous treatment is not considered standard clinical practice because of the unfavorable benefit/risk profile of the previous failed treatment. By “same class of IO agents”, herein it is meant IO agents targeting the same biological response pathway. For example, IO agents that target the PD-1/PD-L1 axis are in the same class. By “same class of cytoxic agent(s)”, it is meant cytotoxic agents with the same mechanism of action. For example, different classes of cytotoxic agents are alkylating agents, cisplatin derivatives, antimetabolites (such as fluorouracil, gemcitabine and methotrexate), cytotoxic antibiotics (such as doxorubicin), topoisomerase inhibitors (such as irinotecan), or anti-microtubule agents (such as paclitaxel).

Specifically, in patients for whom a previous immunotherapy has failed to provide the desired clinical response, e.g., complete response (CR), partial response (PR) or, even stable disease (SD), re-introducing the same IO agent as a monotherapy is no longer indicated for that patient. For example, the selection of patients that may benefit from re-treatment with IO agents from the same class is not established [Levra et al. Immunotherapy rechallenge after nivolumab treatment in advanced non-small cell lung cancer in the real-world setting: A national data base analysis. Lung Cancer 2020]. More specifically, Martini et al. indicate: “Clinicians should refrain from using multiple PD-1/PD-L1 inhibitors sequentially outside of clinical trials until there is sufficient data to support this practice routinely. Prospective studies that allow prior treatment with PD-1/PD-L1 are needed to validate the efficacy and safety of these drugs in the second line or later setting.” [Martini, D. J., et al. Response to single agent PD-1 inhibitor after progression on previous PD-1/PD-L1 inhibitors: a case series. J. Immunotherapy Cancer 5, 66 (2017). https://doi.org/10.1186/s40425-017-0273-y].

Therefore, when a cancer treatment involving an IO agent fails to provide the desired clinical response, administration of cytoxic or cytostatic systemic agents is generally preferred.

When patients with solid tumoral cancer fail to respond fully to treatment involving RT, the disease may progress and LRR, LRR/M, or oligometastatic disease may occur. For Head and Neck (H&N) cancer patients, the Standard of Care (SOC) is typically salvage surgery.

However, many patients, for example, H&N patients, often do not want to undergo surgery due to irreversible negative impact on their quality of life (QoL), e.g., loss of voice, sense of smell, or vision, or disfiguration. In addition, re-irradiation is often limited because of potential toxicity and reduced RT efficacy. The reduced blood supply to the previously irradiated tissue means the radiation will not be effective, as radiation at low doses requires oxygen in the tissue to help facilitate the destruction of tumor DNA [https://www.spohnc.org/recurrent-and-metastatic-head-and-neck-cancer/]. Similarly, rectal cancer patients suffer a negative impact on QoL after resection surgery.

Overall, there is a high unmet medical need to treat cancer patients who have already received a previous treatment involving RT and/or immunotherapy (possibly, in combination with one or more cytotoxic agent(s) or a molecular targeted therapy, as described above herein) for the same cancer, but thereafter develop recurrent disease and/or disease progression. The previous treatment has generally been directed to the primary tumor.

Specifically, there is a high unmet medical need to treat cancer patients who have received a previous treatment involving RT and/or immunotherapy, and who, at clinical staging, present, for example, LRR or LRR with limited number (1-5) of further metastases, or oligometastatic disease (irrespective of the level of control of the previously treated primary tumor).

Specifically, there is a high unmet medical need to provide a therapeutic solution for cancer patients who, after a previous treatment involving RT, or, RT and immunotherapy, at clinical staging, present LRR in the previously irradiated site, optionally accompanied by a limited number (1-5) of further metastases. For the reasons mentioned above, the efficacy of irradiation (and therefore the associated benefit risk) for these patients may be reduced and surgery may not be recommended because of impact on QoL.

Specifically, there is a high unmet medical need to provide a therapeutic solution for cancer patients who, after a previous treatment involving at least one IO agent (in particular, an ICI like an anti PD-1 or an anti-PDL-1 inhibitor), at clinical staging, have oligometastatic disease (even if the primary tumor is well-controlled). These patients are referred to as IO-resistant (at least for the IO agent used in the previous treatment).

Specifically, there remains a critical unmet medical need to provide these groups of patients with therapeutic solutions that can significantly slow disease progression (for example, stop tumor growth), or increase/improve Progression Free Survival (PFS), or Overall Survival (OS), or cure cancer (i.e., convert the patient into a cancer survivor, as further defined herein below).The present invention provides such a therapeutic solution for these patients, who have had a previous treatment involving RT and/or immunotherapy, but go on to present LRR, or LRR with a 1-5 further metastases (LRR/M), or oligometastatic disease (irrespective of the level of control of the previously treated primary tumor). The present invention thus advantageously offers a solution to prevent disease (cancer) progression toward a widespread metastatic disease state in these patient populations, preferably, curing the patient.

### SUMMARY OF THE INVENTION

The invention concerns nanoparticles and/or aggregates of nanoparticles for use as radioenhancer agents when activated by RT, in combination with at least one IO agent for use in the treatment of cancer in specific groups of patients in need thereof. These patients are LRR or LRR/oligometastatic (LRR/M) or oligometastatic cancer patients, who have had a previous treatment involving RT and/or immunotherapy and need further anti-cancer treatment for the same disease.

Thus, the treatment described herein involves, in some cases, re-introducing/re-using at least one element of the previous therapy (RT and/or IO). In a preferred embodiment of the invention, in which the previous treatment involved immunotherapy, the re-introduced/re-used IO agent is in the same class as the IO agent administered in the previous immunotherapy.

Generally, the invention relates to HfO₂ nanoparticles or ReO₂ nanoparticles and any mixture thereof, and/or aggregates thereof for use in the treatment of cancer, typically solid tumoral cancer, in a human patient who has had a previous anti-cancer treatment involving radiotherapy (RT) and/or the administration of at least one immuno-oncology (IO) agent, for the treatment of, preferably, a primary tumor, for the same cancer, but who has, at clinical staging:
(i) at least one loco-regional recurrent (LRR) (cancerous) tumor/lesion (both terms being used indifferently to designate a population of cells comprising cancerous cells), in a previously irradiated site (through RT), and optionally, 1-5 further metastases, or,
(ii) 1-5 metastases (irrespective of the level of control of the previously treated primary (cancerous) tumor/lesion).

According to an embodiment of the invention, in step (a) the nanoparticles and/or aggregates of nanoparticles are administered to only one tumor/lesion or metastasis.

The nanoparticles and/or aggregates of nanoparticles comprise more than 30% by weight of at least one chemical element having an atomic number (Z) between 20 and 83, preferably HfO₂ nanoparticles or ReO₂, and any mixture thereof. The treatment involves a step (a) of administering the nanoparticles and/or aggregates of nanoparticles to at least one, preferably only one, tumor/lesion or metastasis in the patient, a step (b) of exposing the patient who has been administered with the nanoparticles and/or aggregates of nanoparticles to ionizing radiation, and a step (c) of administering at least one IO agent to the patient.

According to an embodiment of the invention, the patient has had a previous anti-cancer treatment involving RT (for example, radiotherapy alone, or radiotherapy combined with a cytotoxic agent, i.e., radiochemotherapy), or RT and immunotherapy, and, at clinical staging, has at least one loco-regional recurrent (LRR) tumor in a previously irradiated site, and, optionally, 1-5 further metastases.

According to an embodiment of the invention the patient to be treated has had previous anti-cancer treatment involved immunotherapy, and, at clinical staging, has 1-5 metastases, irrespective of the level of control of the previously treated primary tumor.

According to an embodiment of the invention, the patient suffers from bladder cancer, metastatic melanoma, (squamous) non-small cell lung cancer (NSCLC), (metastatic) small cell lung cancer (SCLC), (metastatic) head and neck squamous cell cancer (HNSCC), metastatic

Urothelial carcinoma, microsatellite Instability (MSI)-high or mismatch repair deficient (dMMR) metastatic solid tumor cancer (including colorectal cancer), metastatic gastric cancer, metastatic esophageal cancer, metastatic cervical cancer, metastatic Merkle cell carcinoma, and has 1-5 metastases. In a particular aspect, this patient is a patient suffering from solid tumoral cancer for which radiotherapy in combination with immunotherapy using an anti PD-1 inhibitor(s) or anti-PDL-1 inhibitor is indicated, or a patient identified as an anti PD-1 inhibitor non-responder or an anti-PDL1 inhibitor non-responder, and/or for whom monotherapy using an anti PD-1 inhibitor or an anti-PDL1 inhibitor is not indicated.

According to an embodiment of the invention, the IO agent administered in the “previous anti-cancer treatment involving immunotherapy”, is at least one immune check point inhibitor (ICI).

This ICI is preferably selected from an anti PD-1 inhibitor, an anti PDL-1 inhibitor, an anti CTLA-4 inhibitor, and any mixture thereof.

According to an embodiment of the invention, the IO agent used in the context of the present invention, for example, in the herein described step c) is at least one immune check point inhibitor (ICI). This ICI is preferably selected from an anti PD-1 inhibitor, an anti PDL-1 inhibitor, an anti CTLA-4 inhibitor, and any mixture thereof.

According to another embodiment of the invention, at clinical staging, the patient has recurrent head and neck squamous cell carcinoma (HNSCC) LRR that is or is not accompanied by 1-5 further metastases. In a particular aspect, at least one of the metastases is to a lymph node from a HNSCC primary tumor.

According to another embodiment of the invention, at clinical staging, the patient has 1-5 metastases in the lung and/or the liver (exclusively or not).

According to an embodiment of the invention, each nanoparticle of the herein described “nanoparticles and/or aggregates of nanoparticles” are inorganic nanoparticles. Preferably, each nanoparticle and/or aggregate of nanoparticles further comprises a biocompatible surface coating.

According to a preferred embodiment of the invention, the nanoparticles are selected from HfO₂ nanoparticles, ReO₂ nanoparticles and any mixture thereof.

Inventors also describe a pharmaceutical composition comprising nanoparticles and/or aggregates of nanoparticles as herein described and a pharmaceutically acceptable carrier or support.

The pharmaceutical composition can advantageously be used in the treatment of cancer in a human patient who has had a previous anti-cancer treatment, preferably to the primary tumor, involving radiotherapy (RT) and/or immunotherapy, but who has, at clinical staging:
(i) at least one loco-regional recurrent (LRR) (cancerous) tumor/lesion in a previously irradiated site, and optionally, 1-5 further metastases, or
(ii) 1-5 metastases (irrespective of the control or level of control of the previously treated primary (cancerous) tumor/lesion),
wherein the treatment of cancer involves at least one step (a) of administering the pharmaceutical composition to at least one, preferably only one, tumor/lesion or metastasis in the patient, at least one step (b) of exposing the patient who has been administered with the nanoparticles and/or aggregates of nanoparticles to ionizing radiation, and at least one step (c) of administering at least one IO agent to the patient.

The description also concerns a kit comprising a pharmaceutical composition comprising nanoparticles and/or aggregates of nanoparticles and a pharmaceutically acceptable carrier or support as herein described and at least one IO agent, preferably selected from an anti PD-1 inhibitor, an anti-PDL-1 inhibitor, an anti-CTLA4 inhibitor/antibody and any mixture thereof. According to a preferred embodiment of the invention, the kit comprises a pharmaceutical composition as herein described, an anti PD-1 or anti PDL-1 inhibitor, and an anti-CTLA4 inhibitor/antibody.

### FIGURES

FIG. 1: Scheme of an illustrative treatment regimen that may be used to treat patients (defined within the text herein) with claimed nanoparticles (NP). Nanoparticle administration begins on Day 1. Nanoparticle visualization may be typically carried out if desired on Day 2. Typically, the patient receives the first RT fraction between one day and two weeks after the nanoparticle administration, thus, between Day 2 and Day 16. The following RT fractions are generally given in the following five to fifteen days, finishing typically on between Day 12 and Day 31. IO agent administration begins typically on anyone of Day 13-32 and finishes between Day 40 and Day 59. “NP” refers to the nanoparticles or aggregates of nanoparticles described herein. The figure is representative of one treatment regimen. Other treatment regimens are possible, for example, wherein the IO agent administration is carried out during the same or overlapping period as that of the RT.

FIG. 2: Preliminary efficacy data from the Phase I clinical trial NCT03589339; The best observed target lesion response, as per Investigator Assessment based on RECIST 1.1 is indicated in this waterfall plot for the 16 evaluable patients. Patients are identified by capital letters on the X axis. Patients represented as grey columns are anti-PD-1 naïve (M, J, N, O, A). Patients represented as black columns are anti-PD-1 non responders (H, U, Q, I, L, E, D, P, C, G and S). Patient A's (from the head and Neck LRR Cohort, PD-1 naïve) treatment and response is described in Example 2. Patient C's (Lung metastases group, Cohort 2, PD-1 non responder) treatment and response is described in Example 3. Patients G and S's (from the Liver metastases group, Cohort 3, PD-1 non responders) treatment and responses are described in Examples 4 and 5 respectively.

FIG. 3. Preliminary efficacy data from the Phase I clinical trial NCT03589339; Anti-PD-1 Non-Responders Follow-up Since Prior IO Treatment (All Patients Treated: n=14). Grey bars: time between prior IO treatment and NBTXR3 injection (pre-study data). Black bars: time between injection of product of Example 1 and date of last survival status, date of last visit, or date of death. Within the bars, the white dot represents the time point when progression with the prior IO treatment was recorded. This swimmer plot shows that clinical benefits are observed in a population of patients who had previously progressed on anti-PD-1 (except patient D), regardless of the time to progression on the previous anti-PD-1 (primary or secondary resistant).

### DETAILED DESCRIPTION

### Definitions

The terms “treatment” or “therapy” refer to both therapeutic and prophylactic or preventive treatment or measures that can significantly slow disease progression (for example, stop tumor growth) or increase/improve Progression Free Survival (PFS) or Overall Survival (OS), or cure a patient (i.e., turn the patient into a cancer survivor, as further defined herein below).

Such a treatment or therapy is intended for a subject in need thereof, typically a human being (also herein identified as a human patient).

In the art and in the context of the present invention, the terms “treatment having curative intent”, “curative treatment” or “curative therapy” refer to a treatment or therapy, in particular, a treatment comprising a radiotherapeutic step, offering to the subject to be treated a curative solution for treating the cancer(s) he/she is affected by, that is, for globally treating said subject [primary tumor(s) as well as corresponding metastatic lesion(s)/metastasis(es)].

In the context of the invention the term “previous treatment” means any anti-cancer therapeutic regimen/protocol previously used for control of primary or metastatic sites of cancer. The previous treatment may be a first-line therapy. It may also be a second-line or further line therapy. Preferably, the previous treatment is a first line therapy.

In the context of the invention, the term “same cancer” refers to the cancer for which the patient was treated in his “previous treatment”. The previous treatment generally included the treatment of the primary tumor. Thus, at some time after said “previous treatment”, i.e., days, weeks, months or years after said “previous treatment”, the cancer has progressed, either leading to an LRR or LRR/M or an oligometastatic state; in the latter state the primary tumor may or may not be well controlled and 1-5 metastases have developed. Thus, the patient may undergo treatment as described herein via administration of the compositions comprising the nanoparticles or aggregate of nanoparticles combined with RT and administration of at least one IO agent, as herein described.

In the context of the present invention the terms “tumor” and “lesion” are used interchangeably to designate a population of cells comprising cancerous cells. In the present text, unless the terms are preceded by the word “benign”, it is understood that the tumor or lesion is cancerous.

In the context of the invention, “distant metastasis” refers to cancer that has spread from the original (primary) tumor to distant organs or distant lymph nodes. Also called distant cancer.

As well known by the skilled person, the terms “palliative treatment” including, in particular, “palliative radiotherapy”, are used for palliation of symptoms and are distinct from “radiotherapy”, i.e., radiotherapy delivered as curative treatment (also herein identified as “curative radiotherapy”). Indeed, palliative treatment is considered by the skilled person as an efficacious treatment for treating many symptoms induced by locally advanced or metastatic tumors, even for patients with short life expectancy.

In the context of the invention, a patient cured of cancer is identified as a “cancer survivor”. Globally, more than 33 million people are now counted as cancer survivors, and in resource-rich countries, such as the United States, extended survival means that more than 67% of patients survive more than 5 years and more than 25% of patients survive more than 15 years. Long-term (i.e., more than 15 years) cancer survivors may be considered ‘cured’ of their cancer [Dirk De Ruysscher et al. Radiotherapy Toxicity. Nature Reviews, 2019, 5].

In the context of the present invention, the evaluation of response criteria, including the terms “partial response” (PR), “complete response” (CR), “overall response” (OR), “Stable disease” (SD) and “progressive disease” (PD), are according to the current international guidelines, for example, RECIST v1.1 guidelines as published in the European Journal of Cancer 45 (2009) (cf. pp. 228-247 “New response evaluation criteria in solid tumors: Revised RECIST guidelines (version 1.1)”).

In the context of the invention, “IO non-responder” may refer to a patient who did not receive a clinical benefit from IO therapy (IO primary non-responder), and also to a patient who had a documented response followed by disease progression (IO secondary non-responder).

In the context of the invention, “IO primary non-responder” refers typically to a patient for whom PD or for whom a stable disease (SD) is observed during a period of less than 6 months while still receiving IO therapy, or within 12 weeks following the administration of the last dose of the IO agent (according to RECIST 1.1 criteria). SD may typically mean tumor stasis according to RECIST 1.1 criteria. The skilled person understands that the length of the periods “6 months” and “12 weeks” cited above may vary according to International criteria, for example, RECIST criteria.

In the context of the invention, “secondary IO non-responder” refers typically to a patient for whom CR, or PR, or a stable disease (SD) observed during a period of more than 6 months, has been reported, followed by disease progression while still receiving IO therapy. The skilled person understands that the length of the periods “6 months” cited above may vary according to International criteria, for example, RECIST criteria.

In the context of the invention, a patient for whom an IO agent is not indicated as monotherapy, is a patient for whom administration of said IO agent alone is not recommended because of the tumor cells' low expression levels of biomarkers in the biological pathway targeted by said IO agent. For example, today, treatment with an anti-PD-1 antibody, as monotherapy, will not be indicated for certain patients because their tumor cells' expression levels of the PD-1 receptor, ligand PD-L1 are considered too low.

In the context of the current invention, the IO agent may be, for example, an ICI, in which case, the IO non-responder may be referred to as an “ICI non-responder”. The definitions given above for “primary IO non responders” and “secondary IO non responders” apply analogously for “primary ICI non-responders” and “secondary ICI non-responders”. ICI non-responders, specifically, anti-PD-1, or anti-PD-L1 non-responders are patients who are resistant to anti-PD-1 or anti-PD-L1 therapy. Thus, an “anti-PD-1 non-responder” refers to a patient who did not demonstrate a sustainable clinical benefit from an administered anti-PD-1 therapy, and includes those who experience PD or SD during a period of less than 6 months while still receiving the anti-PD-1 treatment (primary anti-PD-1 non-responders), as well as those who have had a documented response followed by disease progression (secondary anti-PD-1 non-responders). The groups “primary anti-PD-1 non-responder” and “secondary anti-PD-1 non-responders” may be defined in an analogous way to primary and secondary IO non responders (see above definitions).

An “anti-PD-L1 non-responder” may include primary anti-PD-L1 non-responders and secondary anti-PD-L1 non-responders, the groups being defined analogously to the definitions provided above for primary and secondary IO non responders.

In the context of the invention, an anti-PD-1 non-responder is a patient for whom treatment with an anti-PD-1 agent as monotherapy is not indicated due to their previous treatment failure.

In the context of the invention, a “patient amenable to re-irradiation” designates a patient with a previous occurrence of a solid tumor, who received a previous treatment involving RT for that tumor and who is amenable to receive RT in a further treatment. Typically, the eligibility for re-irradiation is evaluated by the medical team caring for the patient, which includes at least one oncoradiologist. A patient who is eligible and willing to undergo re-irradiation is thus considered “amenable to re-irradiation”.

In the context of the invention, “a tumor” or “lesion” refers to a cancerous tumor or cancerous lesion. The tumor/lesion may be a primary tumor or a metastatic tumor.

### Patient Group

The patients identified in the present invention are solid tumoral cancer patients having oligometastatic, or loco-regional recurrent (LRR), or LRR accompanied by a limited number further metastases (LRR/M), who have had previous treatment involving RT and/or immunotherapy for the same cancer, typically, for the primary tumor, and who, if they have received RT in the previous treatment, are amenable to re-irradiation. As indicated above, “oligometastatic disease” means having 1-5 metastases.

By treatment “involving RT and/or immunotherapy” it is meant that the previous treatment may have involved RT, or RT and immunotherapy, or immunotherapy. The term “treatment involving” means that the treatment may have comprised other anti-cancer treatments, for example, chemotherapy or targeted molecular therapy.

Generally, the previous treatment may have been administered to the patient, in the previous weeks, months, or years, typically in the previous months or years.

The patients who received a previous anti-cancer treatment involving immunotherapy and did not experience a sustainable CR, PR or even SD may be referred to “IO-non responders”, for example, “ICI-non responders” or “anti-PD-1 non-responders” as defined above, depending on the IO agent received in the previous treatment.

According to a preferred embodiment of the invention, the patient is an “anti-PD-1 non-responder” as defined above. This is, typically, a patient for whom monotherapy with an anti-PD-1 inhibitor is not indicated, due to their previous treatment failure.

According to another preferred embodiment of the invention, the patients is an “anti-PD-L1 non-responder”, as defined above.

According to an embodiment of the invention, the last dose of the previous IO treatment, has generally been administered at least 6 weeks before starting the administration of nanoparticles according to a method or use as herein described. The period of 6 weeks is cited herein as a typical period necessary for systemic washout of the previous immunotherapy. Thus, this period may vary according to the patient and the clearance rate of the previously administered IO agent. Typically, primary IO non-responders are eligible to begin administration of nanoparticles' composition after they are determined to be IO primary non-responders. The composition administration may typically begin 4 weeks to 6 months after their previous immunotherapy treatment started. This period typically includes the time for patient screening that includes systemic washout of the IO agent used in the previous immunotherapy.

In the context of the invention, secondary IO non-responders are typically eligible to begin administration of nanoparticles as soon as disease progression has been diagnosed. The treatment may start after a period sufficient for patient screening and systemic washout of the IO agent used in the previous immunotherapy.

According to a first particular aspect of the invention, the patient has had a previous anti-cancer treatment involving radiotherapy (RT) to at least one solid tumor, or RT combined immunotherapy, but has, at clinical staging, at least one loco-regional recurrent (LRR) tumor/lesion in a previously irradiated site (i.e., in a cancerous site previously exposed to RT), optionally accompanied by 1-5 further metastases, in particular, distant metastases.

The skilled person understands that an LRR tumor is considered a metastatic tumor, and therefore, in the context of the current description, the other metastases observed in the same patient may be referred to as “further” metastases. The skilled person understands that “distant metastases” refers to cancer that has spread from the original (primary) tumor to distant organs or distant lymph nodes.

If said previous cancer treatment comprised immunotherapy, the previous immunotherapy may have occurred before, after, or simultaneously with the previous RT treatment, preferably before, or after previous RT. The previous therapy may have included administration of another anti-cancer treatment (i.e., not RT or treatment with an IO agent), including chemotherapy, which may have been administered before, after, or simultaneously with the RT, or RT and IO, or IO.

Thus, according to this first particular aspect of the invention, the patient has solid tumoral cancer with LRR or LRR with further metastases, and has had a previous anti-cancer treatment involving RT or RT and immunotherapy for the cancer, and is amenable to re-irradiation.

According to an embodiment of this first aspect of the invention, the patient has at least one LRR tumor and between one and five accompanying malignant lesion(s), typically a metastasis/metastases, in particular, a at least one metastatic lymph-node.

According to an embodiment of this first aspect of the invention, patient has inoperable LRR, or LRR/M head and neck squamous cell carcinoma (HNSCC) and is amenable to re-irradiation. The HNSCC may be at stage II, III or IV. For example, the patient may suffer from HNSCC LRR with, additionally, at least one malignant lymph node. Thus, the patient may typically have a lymph node from a HNSCC primary tumor.

According to an embodiment of the first aspect of the invention, the patient may be a patient for whom immunotherapy with a particular IO agent, for example anti-PD-1 antibody or an anti-CTLA-4 antibody, as monotherapy, is not indicated (as defined herein above),

According to a second aspect of the invention, the patients are solid tumoral cancer patients with oligometastatic cancer, irrespective of the level of control of the previously treated primary tumor, and whose previous treatment involved immunotherapy. Thus, following the previously administered treatment, the patient's primary tumor may be fully controlled, partially controlled or not controlled. These patients may suffer from any solid tumoral cancer.

According to an embodiment of this second aspect of the invention, the patient is an ICI-non-responder, preferably an anti-PD-1 or an anti-PD-L1 non responder. According to an embodiment of the second aspect of the invention, the patient may be, typically, a patient with a metastatic lung cancer from any primary solid tumor, or a metastatic liver cancer from any primary tumors, with one to five metastases (oligometastatic disease), preferably, located in the lung or in the liver.

According to an embodiment of the second aspect of the invention, the patient may be a patient for whom an immunotherapy with a particular IO agent, for example an anti-PD-1 antibody or an anti-CTLA-4 antibody is not indicated (as defined herein above).

In the context of the present description, the cancer to be treated may be a solid tumoral cancer that can be, or derive from a cancer selected from, for example, skin cancer, central nervous system cancer, head and neck cancer, lung cancer, kidney cancer, breast cancer, gastrointestinal cancer (GIST), prostate cancer, liver cancer, colon cancer, rectum cancer, anal cancer, esophagus cancer, male genitourinary cancer, gynecological cancer, adrenal and retroperitoneal cancer, sarcomas of bone and soft tissue, pediatric cancer, neuroblastoma, pancreatic cancer and Ewing's sarcoma.

For example, the patient may suffer from one of the following cancers, wherein any metastases are limited in number to between one and five: metastatic melanoma, metastatic non-small cell lung cancer (NSCLC), metastatic small cell lung cancer (SCLC), head and neck squamous cell cancer (HNSCC), metastatic Urothelial Carcinoma, microsatellite Instability (MSI)-high or mismatch repair deficient (dMMR) metastatic solid tumors (including colorectal cancer), metastatic gastric cancer, metastatic oesophageal cancer, metastatic oesophageal junction adenocarcinoma, metastatic squamous cell cancer (SCC) such as metastatic oesophageal squamous cell cancer, metastatic oesophageal cancer, metastatic tumor mutational burden (TMB)-high cancer, metastatic cervical cancer or metastatic Merkle cell cancer/ carcinoma.

According to one embodiment of the invention, the patient is suffering from head and neck squamous cell carcinoma (HNSCC), preferably LRR or LRR/M HNSCC wherein the metastases, if present, are limited in number to between one and five.

According to one embodiment of the invention, the patient is suffering from (metastatic) non-small cell lung carcinoma (NSCLC), or (metastatic) small cell lung carcinoma (SCLC), wherein the metastases, if present, are limited in number to between one and five.

According to an embodiment of the invention, the patient is suffering from any solid tumoral cancer for which treatment with ICI(s) combined with radiotherapy is clinically approved.

According to an embodiment of the invention, the patient has a solid tumoral cancer and radiotherapy combined with immunotherapy using anti-PD-1 or anti-PD-L1 inhibitor(s) is indicated for said patient.

According to an embodiment of the invention, the patient has a solid tumoral cancer and radiotherapy combined with immunotherapy using anti-PD-1 or anti-PD-L1 inhibitor(s) combined with an anti-CTLA4 inhibitor is indicated for said patient.

According to an embodiment of the invention, the patient is suffering from a solid tumoral cancer for which immunotherapy using anti-PD-1 or anti-PD-L1 inhibitor(s) combined with radiotherapy is clinically approved, for example bladder cancer, metastatic melanoma, (squamous) NSCLC, (metastatic) SCLC, (metastatic) HNSCC, metastatic Urothelial carcinoma, MSI-high or dMMR metastatic solid tumors (including colorectal cancer), metastatic gastric cancer, metastatic esophageal cancer, metastatic cervical cancer, or metastatic Merkle cell carcinoma, and wherein the metastases are limited in number to between one and five.

According to an embodiment of the invention, the patient is suffering from rectal cancer, the metastases being limited in number to between one and five. According to an embodiment of the invention, the patient is suffering from lung cancer, the metastases being limited in number to between one and five. According to an embodiment of the invention, the patient is suffering from thyroid cancer, the metastases being limited in number to between one and five. According to an embodiment of the invention, the patient is suffering from bladder cancer, the metastases being limited in number to between one and five. According to an embodiment of the invention, the patient is suffering from head and neck cancer, the metastases being limited in number to between one and five.

According to an embodiment of the invention, the patient is suffering from melanoma cancer, the metastases being limited in number to between one and five. According to an embodiment of the invention, the patient is suffering from gastric cancer, the metastases being limited in number to between one and five. According to an embodiment of the invention, the patient is suffering from esophageal cancer, the metastases being limited in number to between one and five. According to an embodiment of the invention, the patient is suffering from cervical cancer, the metastases being limited in number to between one and five. According to an embodiment of the invention, the patient is suffering from urothelial cancer, the metastases being limited in number to between one and five.

According to one embodiment of the invention, the patient to be treated may be any patient suffering from any solid tumoral cancer for whom radiotherapy in combination with immunotherapy, preferably an anti-PD1 inhibitor and/or an anti-PDL-1 inhibitor, is indicated.

According to one embodiment of the invention, the patient to be treated may be any patient suffering from any solid tumoral cancer, for whom a monotherapy treatment with an anti-PD1 inhibitor or an anti-PDL-1 inhibitor is not indicated.

According to an embodiment of the invention, the patient is suffering from any solid tumoral cancer for which treatment using anti-CTLA-4 inhibitor(s) in combination with radiotherapy is indicated.

### Immuno-Oncology (IO) Agent to be Administered

In the context of the present invention, the at least one IO agent to be administered is typically one that has been approved for clinical use, preferably for the cancer from which the patient suffers. As mentioned above, the patient may also be a patient for whom administration of an IO agent as monotherapy is not indicated based on the patient's insufficient tumor cell levels of biomarkers related to the pathway targeted by said IO agent. The patient may also be a patient previously identified as a non-responder to said IO agent. Thus, said IO agent is not indicated as a monotherapy for said non responder. Without being bound by theory, the inventors consider that the combination of the nanoparticles or aggregates of nanoparticles activated by ionizing radiation and at least one IO agent provides an improved anti-cancer response, i.e., improved cell killing compared to administration of the IO agent alone or the IO agent with RT.

According to an embodiment of the invention, the IO agent to be administered may be selected from a monoclonal antibody, a cytokine and a combination thereof.

According to an embodiment of the invention, the IO agent to be administered is an immune check point inhibitor (ICI).

According to an embodiment of the invention, the IO agent to be administered is an antibody selected from an anti-CTLA-4 antibody, an anti-PD-1 antibody, an anti-PD-L1 antibody, an anti-PD-L2 antibody; a monoclonal antibody enhancing CD27 signaling, CD137 signaling, OX-40 signaling, GITR signaling and/or WWII signaling and/or activating CD40; a monoclonal antibody inhibiting TGF-f3 signaling or KIR signaling; a cytokine selected from granulocyte-macrophage colony stimulating factor (GM-CSF), a fms-related tyrosine kinase 3 ligand (FLT3L), IFN-α, IFN-α2b, IFNg, IL2, IL-7, IL-10 and IL-15; an immunocytokine; an immune cell presenting, or sensitized to, a tumor antigen; a cell secreting an immunogenic molecule; a dead tumor cell or a dying tumor cell expressing CRT and/or producing HMGB1 and/or producing ATP in a ICD amount; or a Toll-like receptor agonist selected from a TLR 2/4 agonist, a TRL 7 agonist, a TRL 7/8 agonist and a TRL 9 agonist.

According to an embodiment of the invention, the IO agent to be administered is an antibody selected from an anti-CTLA-4 antibody, an anti-PD-1 antibody, an anti-PD-L1 antibody and any mixture thereof.

According to an embodiment of the invention, the IO agent to be administered is an anti PD-1 antibody selected from Nivolumab, Pembrolizumab, Cemiplimab, Spartalizumab, Camrelizumab, Sintilimab, Tislelizumab, Toripalimab, Dostarlimab, INCMGA00012, AMP-224 and AMP-514.

According to an embodiment of the invention, the IO agent to be administered is an anti-PD-L1 antibody selected from Atezolizumab, Avelumab, Aurvalumab, Durvalumab, Atezolizumab, KN035, CK-301, AUNP12, CA-170 and BMS-986189.

According to an embodiment of the invention, the IO agent to be administered is an anti-CTLA-4 antibody, preferably ipilimumab or tremelimumab.

According to an embodiment of the invention, the IO agent to be administered is an anti-CD40 antibody, for example dacetuzumab or lucatumumab.

According to an embodiment of the invention, the at least one IO agent to be administered is an anti-CD137 antibody, for example urelumab. The latter antibody is currently in trials to treat metastatic solid tumors, NSCLC, melanoma, B-cell non-Hodgkin lymphoma, colorectal cancer or multiple myeloma.

According to an embodiment of the invention, the IO agent to be administered is an anti-TGF-antibody, for example, fresolimumab. The latter antibody is used to treat kidney cancer and melanoma.

According to an embodiment of the invention, the IO agent to be administered is an antibody targeting a Killer-cell immunoglobulin-like receptor (KIR), for example lirilumab that is currently in clinical trials to treat HNSCC.

According to an embodiment of the invention, the IO agent to be administered is a Toll-like receptor agonist selected from imiquimod, bacillus Calmette-Guérin and monophosphoryl lipid A.

According to an embodiment of the invention, the IO agent to be administered is an immunocytokine such as, for example, anyone of the following immunocytokines: interleukin [IL]-2, tumor necrosis factor [TNF]-α, interferon [IFN]-α2, granulocyte-macrophage colony-stimulating factor [GMCSF], or any combination thereof.

According to an embodiment of the invention, more than one IO agent are administered during the step c) of administration of at least one IO agent. The IO agents may be from the same class (e.g., both IO agents are ICIs) or from different classes (e.g., one is an ICI and the other is an immunocytokine).

Within the same class, the IO agents may have the same or different mechanisms of action. According to one embodiment of the invention, the IO agents are anti-PD-1/PDL-1 inhibitors acting on the same signaling pathway. According to one embodiment of the invention, the IO agents are ICIs, but acting on different signaling pathways. For example, at least one is an anti-PD-1/PDL-1 inhibitor and at least one is an anti-CTLA-4 inhibitor.

For example, according to one embodiment of the invention, the at least one IO agent to be administered to the patient is an anti-CTLA-4 antibody and an anti-PD-1 antibody (or an anti PDL-1 antibody). According to one embodiment of the invention, a first “priming” dose of anti-CTLA-4 antibody is administered to the patient, followed by at least one dose of at least one anti-PD-1 antibody (or an anti PDL-1 antibody).

Other examples of ICIs that may be administered in the context of the invention are antagonists/inhibitors of the following receptors: GITR, 4-BB, CD27, TIGIT, LAG3, TCR, CD40L, OX40 and/or CD28 and inhibitors of their respective natural ligands.

Alternatively, the IO agents to be administered to the patient may be from different classes, for example, at least one ICI and at least one anti-KIR.

The medical team treating the patient selects the most appropriate combination of IO agents for said patient, given the type and stage of cancer to be treated as well as the patient's capacity to undergo treatment.

In the case of administration of more than one IO agent, the different IO agents may be administered concurrently or sequentially or during steps that are partially concurrent and partially sequential, depending on the clinical protocol used for each patient and according to the standard clinical practice known to the medical team looking after the patient.

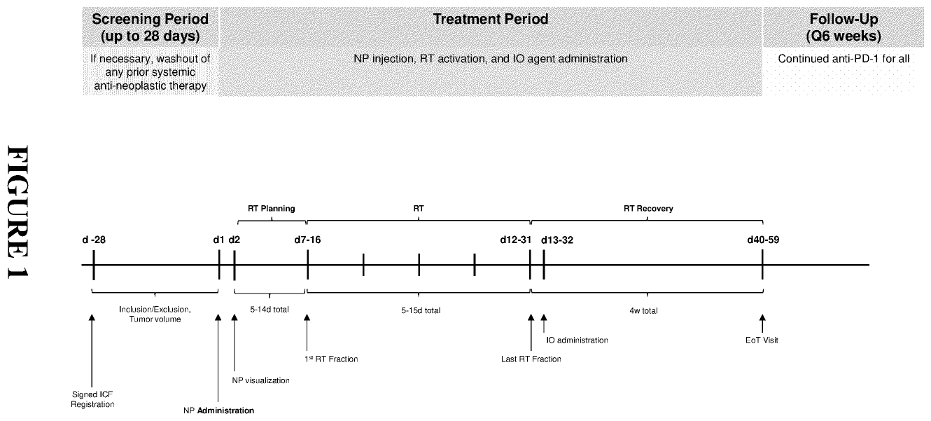

According to an embodiment of the invention, the at least one IO agent may be administered to the human patient, either simultaneously with, or after the administration of the nanoparticles or aggregates of nanoparticles. Typically, the IO agent is administered between 2 to 14, preferably 7 to 14 days, more preferable between 12 to 14 days, after the administration of the nanoparticles or aggregates of nanoparticles (see FIG. 1 for a typical clinical protocol that may be used for the current invention).

### Nanoparticles and/or Aggregates of Nanoparticles

### Size

In the context of the invention, the term “nanoparticle” refers to a product, in particular, a synthetic product, with a size in the nanometer range, typically between about 1 nm and about 1000 nm, preferably between about 1 nm and about 500 nm, even more preferably between about 1 and about 100 nm.

The term “aggregate of nanoparticles” refers to an assemblage of nanoparticles.

The size of the nanoparticle and/or aggregates of nanoparticle can typically be measured by Electron Microscopy (EM) technics, such as transmission electron microscopy (TEM) or cryo-TEM, as well known by the skilled person. The size of at least 100 nanoparticles and/or aggregates of nanoparticles is typically measured and the median size of the population of nanoparticles and/or aggregates of nanoparticles is reported as the size of the nanoparticle and/or aggregate of nanoparticles.

### Shape

As the shape of the nanoparticles and/or aggregates of nanoparticles can influence its “biocompatibility”, nanoparticles and/or aggregates of nanoparticles having a quite homogeneous shape are preferred. For pharmacokinetic reasons, nanoparticles and/or aggregates of nanoparticles being essentially spherical, round or ovoid in shape are thus preferred. Such a shape also favors the nanoparticle and/or aggregates of nanoparticles interaction with, or uptake by, cells.

### Composition/Structure

In a preferred aspect herein described the nanoparticles and/or aggregates of nanoparticles of the present invention comprise more than 30%, preferably more than 40%, 50%, 60%, 70% or 80% by weight of HfO₂ nanoparticles or ReO₂ nanoparticles or any mixture thereof. The nanoparticles may be discrete nanoparticles of HfO₂ or discrete nanoparticles of ReO₂, or discrete nanoparticles of a mixture of HfO₂ and ReO₂. Similarly, the aggregates of nanoparticles may be aggregates of nanoparticles of HfO₂, or aggregates of nanoparticles of ReO₂, or aggregates of a mixture of HfO₂ and ReO₂ nanoparticles.

The determination of the percentage of HfO₂ nanoparticles or ReO₂ nanoparticles is performed on the nanoparticles and/or aggregates of nanoparticles having no biocompatible surface coating as herein below described (i.e., prior any biocompatible surface coating of the nanoparticle and/or aggregate of nanoparticles), typically using an Inductively Coupled Plasma (ICP) source, such as an ICP-MS (Mass Spectroscopy) tool, or an ICP-OES (Optical Emission Spectroscopy) tool. The results of the quantification are typically expressed as a percentage (%) by weight of the chemical element per weight of the nanoparticle and/or aggregate of nanoparticles (i.e., % w/w).

As a theoretical example, if the nanoparticle and/or aggregate of nanoparticles is made of hafnium oxide (HfO₂), the theoretical percentage (%) by weight of the chemical element hafnium (Hf) (Z_{Hf}=72) per weight of the nanoparticle and/or aggregates of nanoparticles (hafnium oxide (HfO₂)) is equal to 85% (% w/w):

178.49/210.49×100=85% (% w/w), where 178.49 is the molecular weight of Hf element and 210.49 is the molecular weight of HfO₂ material.

Any experimental quantification of a chemical element constituting the nanoparticle and/or aggregate of nanoparticles can be expressed as a percentage by weight of this chemical element per weight of nanoparticle and/or aggregate of nanoparticles as herein above presented in the context of a theoretical calculation.

The inorganic material of the nanoparticle and/or aggregate of nanoparticles preferably has a theoretical (bulk) density of at least 7 g/cm³ and may be selected from any material exhibiting this property and identified in the table from Physical Constants of Inorganic Compounds appearing on page 4-43 in Handbook of Chemistry and Physics (David R. Lide Editor-in-Chief, 88th Edition 2007-2008).

### Biocompatible Coating

In a particular aspect of the invention, each of the nanoparticles and/or aggregates of nanoparticles of the present invention further comprises a biocompatible surface coating.

In a preferred aspect, each of the nanoparticle and/or aggregate of nanoparticles used in the context of the present invention can be coated with a biocompatible material, preferably with an agent exhibiting stealth property. Indeed, when the nanoparticles and/or aggregates of nanoparticles of the present invention are administered to a subject via the intravenous (IV) route, a biocompatible coating with an agent exhibiting stealth property is particularly advantageous to optimize the biodistribution of the nanoparticles and/or aggregates of nanoparticles. Such coating is responsible for the so called “stealth property” of the nanoparticle or aggregate of nanoparticles. The agent exhibiting stealth properties may be an agent displaying a steric group. Such a group may be selected for example from polyethylene glycol (PEG); polyethylenoxide; polyvinylalcohol; polyacrylate; polyacrylamide (poly(N-isopropylacrylamide)); polycarbamide; a biopolymer; a polysaccharide such as for example dextran, xylan and cellulose; collagen; and a zwitterionic compound such as for example polysulfobetain; etc.

In another preferred aspect, each of the nanoparticle and/or aggregate of nanoparticles can be coated with an agent allowing interaction with a biological target. Such an agent can typically bring a positive or a negative charge on the nanoparticle's or aggregate of nanoparticles' surface. This charge can be easily determined by zeta potential measurements, typically performed on nanoparticles and/or aggregates of nanoparticles suspensions the concentration of which vary between 0.2 and 10 g/L, the nanoparticles and/or aggregates of nanoparticles being suspended in an aqueous medium with a pH comprised between 6 and 8.

An agent forming a positive charge on the nanoparticle' s or the aggregate of nanoparticles' surface can be for example aminopropyltriethoxisilane or polylysine. An agent forming a negative charge on the nanoparticle' s or the aggregate of nanoparticles' surface can be for example a phosphate (for example a polyphosphate, a metaphosphate, a pyrophosphate, etc.), a carboxylate (for example citrate or dicarboxylic acid, in particular succinic acid) or a sulphate.

A typical example of a nanoparticle according to the invention is a nanoparticle made of HfO₂ or ReO₂ comprising a phosphate compound such as sodium trimetaphosphate (STMP) or sodium hexametaphosphate (HMP) as a biocompatible coating.

The biocompatible coating allows, in particular, the nanoparticle's and/or aggregate of nanoparticles' stability in a fluid, typically in a physiological fluid (such as blood, plasma, serum, etc.), and in any isotonic media or physiologic media, for example any media comprising glucose (5%) and/or NaCl (0.9) which may be used in the context of a pharmaceutical administration.

Stability may be confirmed by dry extract quantification and measured in a suspension of nanoparticles and/or aggregates of nanoparticles prior and after filtration, typically on a 0.22 μm or 0.45 μm filter. Advantageously, the coating preserves the integrity of the nanoparticle and/or aggregate of nanoparticles in vivo, ensures or improves the biocompatibility thereof, and facilitates an optional functionalization thereof (for example, with spacer molecules, biocompatible polymers, targeting agents, proteins, etc.).

### Targeting

A particular nanoparticle and/or aggregate of nanoparticles as herein described further comprise a targeting agent allowing its interaction with a recognition element present on a target cell, typically on a cancer cell. Such a targeting agent typically acts once the nanoparticles and/or aggregates of nanoparticles are accumulated on the target site, typically on the tumor site. The targeting agent can be any biological or chemical structure displaying affinity for molecules present in the human or animal body. For instance, it can be a peptide, oligopeptide or polypeptide, a protein, a nucleic acid (DNA, RNA, SiRNA, tRNA, miRNA, etc.), a hormone, a vitamin, an enzyme, the ligand of a molecule expressed by a pathological cell, in particular the ligand of a tumor antigen, hormone receptor, cytokine receptor or growth factor receptor. Said targeting agent can be selected for example in the group consisting in LHRH, EGF, a folate, an anti-B-FN antibody, E-selectin/P-selectin, anti-IL-2Rα antibody, GHRH, etc.

### Composition

Also herein described is a pharmaceutical composition comprising nanoparticles and/or aggregates of nanoparticles such as herein above described, and a pharmaceutically acceptable carrier, vehicle, or support. The said pharmaceutical composition is suitable for use in the treatment of cancer as described herein above.

### Administration of Nanoparticles or Aggregates of Nanoparticles or of the Composition Comprising Them

The nanoparticles or aggregates of nanoparticles as herein described or the composition comprising such nanoparticles or aggregates of nanoparticles are advantageously administered to the patient before RT is administered. The administration can be performed by administration to the patient directly into the tumor, tumor bed (after tumor resection by surgery) or tumor metastasis. The administration can be carried out using different possible routes such as local [intra-tumoral (IT), intra-arterial (IA)], subcutaneous, intra venous (IV), intra-dermic, airways (inhalation), intra peritoneal, intramuscular, intra-articular, intrathecal, intra-ocular or oral route (per os), preferably using IT, IV or IA.

Generally, the administration of the nanoparticles and/or aggregates of nanoparticles or composition comprising same, is to at least one, tumor or lesion in the patient, who has either an LRR (cancerous) tumor, LRR (cancerous) tumor with metastases (LLR/M) or an oligometastatic disease state/cancer. Preferably, said administration is to only one tumor/lesion in said patient. As discussed above, the current approach for treating oligometastatic or LRR/M patients favors treatment of multiple local sites combined with a systemic treatment. Administering local RT treatment to only one tumor/lesion combined with administration of a systemic I/O agent as herein taught thus goes against the current approaches.

However, surprisingly, the inventors have observed, in preliminary results, that, for at least two patients (patients J et C), injection of the nanoparticles and/or aggregates of nanoparticles according to the invention into only one site resulted in tumor/lesion shrinkage in all non-injected sites, some of which had not received any radiation. The observed effect may be termed an “abscopal effect” and has the impact of reducing overall tumor burden with limited medical intervention to the patient.

According to an embodiment of the invention, repeated injections or administrations of nanoparticles into the same tumor/lesion can be performed, when appropriate.

### Ionizing Radiation

The ionizing radiation used may be selected from X-rays, gamma-rays, electrons and protons.

Methods of radiation that may be used in the context of the current invention include conventional RT, accelerated fractionation (i.e., compared to conventional RT, generally, the same total dose is delivered but in a shortened treatment time) and hyperfractionation (i.e., compared to conventional RT, generally, a higher total dose is delivered in the same treatment time, typically twice daily), so that the killing effects on the tumor exceed those on normal tissues. Furthermore, radiation regimens involving a relatively large radiation dose per fraction (i.e., up to typically 20 Gy or 25 Gy) and highly conformal techniques may be used. With these regimens, known as stereotactic body radiation therapy (SBRT) (also called stereotactic ablative radiotherapy (SABR)), ablative doses are delivered over a short period, typically, 1 to 2 weeks.

According to an embodiment of the invention, the RT used is FLASH RT therapy as described, for example, in Symonds and Jones (2019) “FLASH Radiotherapy: The Next technological Advance in Radiation therapy?” Clin. Oncol. 31, 405e406.

According to one embodiment of the invention, the total radiation dose delivered in the treatment concerned by the invention is higher than that used typically for palliative care (e.g., total dose of 8, 10, 12, 14 or 16 Gy). However, in other embodiments of the invention, doses that are currently used in palliative radiation may be used because the presence of the nanoparticles allows a local increase in radiation dose deposit in the cells. Thus, the patient may be able to withstand the RT to a better extent compared to doses typically used for curative RT and the heathy tissue surrounding the tumor is spared to a greater degree.

According to one embodiment of the invention, conventional radiation techniques may be used in the RT. For example, the treatment may comprise at least one irradiation step wherein the ionizing radiation dose ranges from 5 to 20 Gray (Gy), preferably 7 to 15 Gray (Gy), typically 7 or 8, 9, 10, 11, 12, 13, 14, 15 Gray (Gy), with a total dose of at least 20 Gy, preferably of at least 25 Gy.

According to one embodiment of the invention, the total ionizing radiation dose given during the treatment may ranges from 25 to 80 Gray (Gy), preferably 30 to 70 Gray (Gy), typically from 30 to 45 Gray (Gy).

According to one embodiment of the invention, fractionated stereotactic body radiation therapy (SBRT) is used.

According to one embodiment of the invention, fractionated radiotherapy with three to seven fractions is used comprising at least one irradiation step wherein the total ionizing radiation dose ranges from 25 to 60 Gray (Gy), preferably 30 to 50 Gray (Gy), typically from 35 to 45 Gray (Gy). The radio-oncologist treating the patient may adjust the radiation doses appropriately in view of the disease state and the patient's capacity to undergo radiation.

According to one embodiment of the invention, the fractionated RT is delivered as five fractions of 7 Gy. According to one embodiment of the invention, the fractionated RT is delivered as five fractions of 9 Gy. According to one embodiment of the invention, the fractionated RT is delivered as three fractions of 15 Gy.

Generally, if RT was used in the previous treatment, the specific type of RT treatment may be the same as or different from the RT treatment used in the previous treatment.

Generally, on Day 1 of the treatment, the patient receives an injection of a composition comprising the nanoparticles and/or aggregates of nanoparticles. Generally, the patient then receives a first RT dose, for example, from between one day to 14 days, between one day and 7 days, between two and ten days, between four and ten days, between four and 12 days, or between one and two weeks after the injection. A further number of RT doses may be delivered during, for SBRT, for example ten days to two weeks following the first dose of RT, for example each day or, every other day, starting on Day 12 and during days 12-35. IO agent administration to the patient may be preferably started soon (e.g., one, two or three days) after RT has finished. IO agent administration may be preferably started between one and 14 days after RT has finished. The clinical team looking after the patient generally decides when the IO administration begins. The necessary number of IO administrations is given to ensure optimal clinical outcome for the patient.

FIG. 1 shows an illustrative treatment protocol that may be used according to an embodiment of the invention. On Day 1, the patient typically receives an injection of a composition comprising the nanoparticles and/or aggregates of nanoparticles. The patient may then receive a first RT dose one to two weeks after the injection. A further number of RT doses may be delivered during days 12-35. IO agent administration may be preferably started one to three days after RT has finished. Optionally, the IO agent administration may be carried out at the same time or during an overlapping period as that of the RT. Thus, according to an embodiment of the invention, IO administration may be in parallel with RT, meaning that the patient receives IO administration in the same period or a period overlapping with that in which he receives the RT.

The patient may be assessed usually between 45-59 days after start of treatment and the response recorded according to the Guidelines RECIST 1.1.

The invention also concerns a kit comprising a pharmaceutical composition comprising nanoparticles and/or aggregates of nanoparticles and a pharmaceutically acceptable carrier or support as herein described and at least one IO agent, preferably selected from an anti-PD-1 inhibitor, an anti-PDL-1 inhibitor, an anti-CTLA4 inhibitor/antibody and any mixture thereof. According to a preferred embodiment of the invention, the kit comprises a pharmaceutical composition as herein described, an anti-PD-1 or anti-PDL-1 inhibitor, and an anti-CTLA4 inhibitor/antibody. The kit comprises suitable containers for each of the components.

### Technical Effect

The technical effect of the invention may be illustrated by the preliminary results from ongoing phase 1 clinical trial NCT03589339, which are disclosed herein for the first time. The trial is an open-label, Phase I, prospective clinical study to assess the safety of intra-tumoral injection of the nanoparticles' composition, described in Example 1 below, activated by radiotherapy in combination with anti-PD-1 therapy, in two groups of cancer patients.

One group of patients had HNSCC for which their previous treatment involving RT was non-curative and, thus, the patients were in a progressive disease (PD) state when they enrolled for the trial. They had loco-regional recurrence (LRR) that was, in some cases, accompanied by a limited number of metastases (generally one or two). The patients were amenable to re-irradiation.

The second group of patients had solid tumor oligometastatic cancer, for which their previous treatment involved administration of an ICI and had proved to be non-curative. These patients either had liver or lung metastases from any primary cancer.

The patients underwent nanoparticles injection and irradiation to one metastatic site. Tumor shrinkage was observed in the injected target sites and, for one patient, also in non-injected sites, some of which had received no radiation at all. This surprising abscopal effect has been documented as an infrequent clinical occurrence. These preliminary results indicate improved clinical outcomes for both patient groups and the absence of any severe adverse effects.

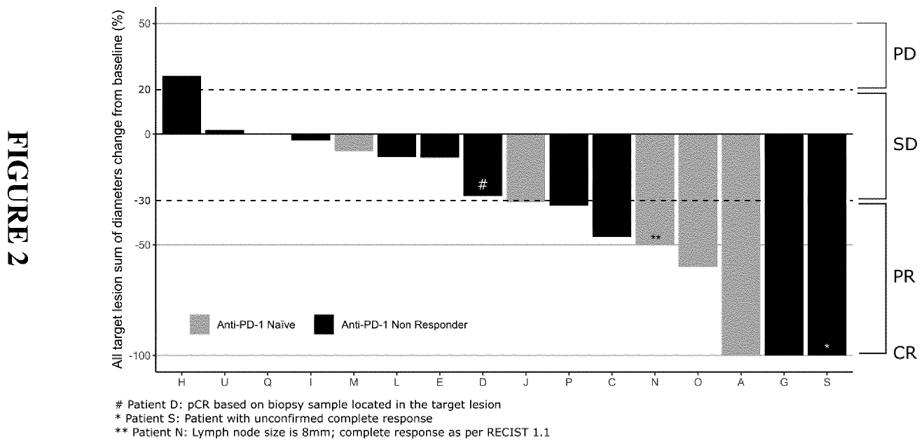

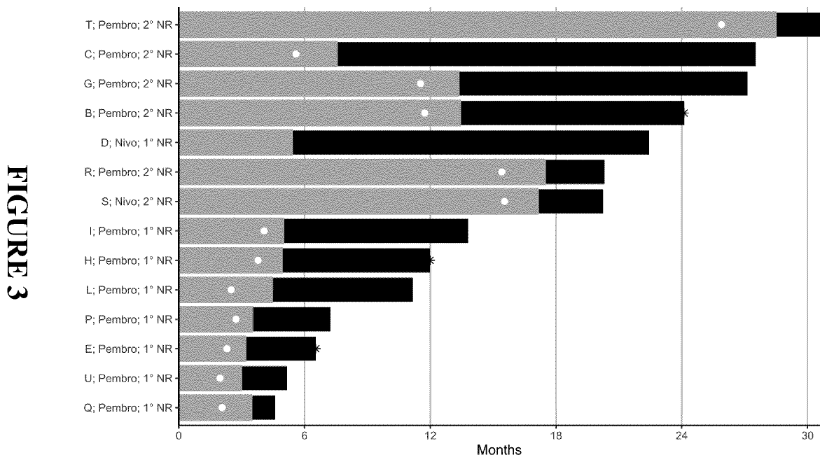

Specifically, FIGS. 2 and 3 summarize the efficacy data from the preliminary results. FIG. 2 (waterfall plot) shows the change in tumor size (from baseline) over time. Responses PD, SD, PD and CR (according to RECIST 1.1 criteria) are indicated on the graph. The grey bars indicate the response for anti-PD-1 naïve patients, while the black bars indicate the response for anti-PD-1 non responder patients.

This Figure demonstrates that tumor regression was observed in 13 out of 16 anti-PD1 naive or non-responder evaluable patients: three anti-PD-1 naïve patients (A, O and N) showed complete response, one anti-PD-1 naïve patient (J) had a partial response, and one anti-PD-1 naïve patient has stable disease (patient M) for over two years. Eight out of eleven anti-PD-1 non-responder patients had post-treatment responses, including a complete response for patient G (see Example 4) and patient S (see example 5). Patient G had liver metastases from a primary HNSCC. Patient S had lung metastases from a primary rectal cancer.

In this study, in three patients that already failed response on prior IO treatment, administration of the nanoparticles' composition and radiotherapy and administration of anti-PD-1 reverse resistance to previous anti-PD-1 treatment. This is a demonstration of the abscopal effect, which is rare in a clinical setting, induced by the administration of the nanoparticles' composition.

Thus, the disease was controlled in two patients (I and L) having highly progressive disease (PD while receiving anti-PD-1 within 6 months of therapy). These patients achieved best observed response of Stable Disease on non-target, non-irradiated lesions.

Reverse resistance was achieved in Patient C (described in Example 3): this patient achieved best observed response of CR in non-target, non-irradiated lesion.

Furthermore, patient G (Example 4) with a liver metastasis from a Stage IV HNSCC with prior secondary resistance, showed a delayed and confirmed response that has deepened over time, with a best observed response (BOR) of CR (−100%) based on RECIST 1.1

The data in FIG. 3 indicate that, according to one embodiment of the invention, clinical benefits are attained in most patients who had previously progressed on anti-PD-1, regardless of the time to progression on the previous anti-PD-1 (primary or secondary resistant).

These surprising data indicate that the intra tumoral administration of the nanoparticles-comprising composition, associated with radiotherapy results in a higher-than-expected positive response to anti-PD-1 among anti-PD1-naive patients, and a surprising positive anti-PD1 response in patients who had been identified as anti-PD1 non-responders.

Thus, the inventors have shown that the inventive treatment results in a positive clinical outcome for specific oligometastatic patients i.e., IO non-responders and patients whose previous treatment involving RT was non-curative in that cancer recurred. The positive clinical outcome achieved by the treatment described herein has been achieved by injecting just one cancerous tumor/lesion.

Thus, the inventors have shown that the one-site (lesion/metastasis) treatment approach according to an embodiment of the present invention, which is very different from a multi-site local treatment approach for treating oligometastatic patients, is safe and offers an innovative therapeutic solution for these specific groups of patients.

The clinical data indicates that the claimed treatment approach demonstrates efficacy at all tested doses and that patients' lives may be prolonged after the initial anti-PD-1 therapy failure. While almost all non-responder patients had previously progressed on anti-PD-1 (only one SD on anti-PD-1), the rate of best objective response indicates that administration of the claimed nanoparticles can reverse resistance to immunotherapy.

The claimed treatment boosts the therapeutic effect of the administered ICI, in particular, anti-PD-1 therapy, in anti-PD-1 naive patients. The claimed treatment also allows anti-PD-1 therapy to become effective in anti-PD-1 non-responders patients. Furthermore, the preliminary data demonstrate the correlation between the local and systemic response in both anti-PD-1-naive and post-anti-PD-1-failure patients. The clinical trial data also show how the treatment can trigger an abscopal effect in non-irradiated lesions.

Other aspects and advantages of the invention will become apparent in the following examples, which are given for purposes of illustration and not by way of limitation.

### EXAMPLES

### Example 1

As an example of the nanoparticles for use according to the current invention, we may cite

Example 1 from published international patent application WO 2016/189125.

### Example 2

Patient A presented with LRR HNSCC Stage III with the cancerous lesion in the lymph node (Cohort 1). The patient's previous RT was more than 6 months prior to the diagnosis of the LRR disease.

On day 1, the patient received an injection of 5.4 ml of the composition of Example 1 into the ml tumor, then experienced a first RT fraction of 8 Gy at day 8 after the injection. A further four fractions of 8 Gy were delivered during days 12-31. An anti-PD-1 inhibitor (200 mg pembrolizumab) was administered by IV route on day 18 and a further 15 doses of pembrolizumab were administered.

The patient was assessed on day 40-59 and the response was recorded as complete response (CR) according to the Guidelines RESCIST v1.1. The confirmed CR has lasted over two years and the patient is currently on follow-up. The patient did not experience any severe adverse effect or dose-limiting toxicity.

### Example 3

Patient C presented with one lung primary tumor and three metastases (one in lung, two in lymph nodes) from stage IV NSCLC (Cohort 2). The patient was tested as PD-L1 positive.

The patient's previous treatment consisted of a combination of chemotherapy and an anti-PD-1 inhibitor (which led to an initial partial response), followed by an anti-PD-1 inhibitor alone which then led to progressive disease. The patient was classified as an anti-PD1 primary non responder.

On day 1, the patient received one injection of 20.9 ml of the composition of Example 1 into one lung metastasis (volume 95.1 ml), then experienced a first RT fraction of 9 Gy at one-two weeks after the injection. A further four fractions of 7 Gy were delivered during days 12-31. An anti-PD-1 inhibitor was administered by IV on day 20 and a further number of anti-PD-1 administrations were given.

The patient's post-treatment follow-up scans (evaluated with RECIST 1.1 criteria) showed a significant decrease (˜45%) in tumor size with confirmed partial response to treatment. Further, a complete response was recorded for the non-target lesions. The patient is no longer on the study (withdrew consent) and is alive, at the time of filing.

### Example 4

Patient G presented with Stage IV HNSCC with liver metastases (Cohort 3). The patient was PD-L1 positive and RT naïve.

The patient's previous treatment consisted of a combination of chemotherapy (carboplatin/paclitaxel/Cetuximab) for four weeks and an anti-PD-1 inhibitor, which lead to an initial complete response, at 7 months followed by disease progression. The patient was therefore considered an anti PD-1 secondary non-responder.

On day 1, the patient received one injection of 1.2 ml of the composition of Example 1 into a 5.3 ml lung metastasis, then received 45 Gy stereotactic body radiation therapy (SBRT) in 3 fractions beginning on day 12 and after the injection. An anti-PD-1 inhibitor (pembrolizumab) was administered by IV on day 19 and a further number of anti-PD-1 administrations were given.

The patient's post-treatment follow-up scans (evaluated with RECIST 1.1 criteria) have shown a confirmed complete response to treatment, the tumor having completely disappeared.

### Example 5

Patient S presented with Stage IV tumor mutation burden high (TMB-H) rectal cancer with lung and bone metastases (Cohort 2). The most recent patient's previous RT was more than six months prior to the study treatment and the most recent administration of an anti-PD1 inhibitor (nivolumab) was one month before the study treatment. On day 1, 12 May 21, the patient received a 1.25 ml of the composition of Example 1 into a 3.8 ml lung metastasis, then experienced a first RT fraction of 9 Gy at day 7 after the injection. A further four fractions of 9 Gy were delivered during days 12-31. An anti-PD1 inhibitor (480 mg nivolumab) was administered by IV route on day 19 and the IO treatment is still ongoing. The patient was assessed on 25 Jun. 21, at End of Treatment (EOT) visit, and the response was recorded as partial response (PR) for both target lesions and overall disease according to RESCIST 1.1. On the next assessment at the first follow up visit (FUP1) on 11 Aug. 21 the response was assessed as complete response (CR) for target lesions but the patient was progressed per non target lesions (NTLs). The patient did not experience any severe adverse effect or dose-limiting toxicity.

### Example 6

Patient N presented with Stage IV metastatic HNSCC with regional lymph node metastases and distant bone and lung metastases (Cohort 2). The most recent patient's previous RT was more than 6 months prior to study treatment and the patient did not receive anti-PD1 treatment before the study. On day 1, 2 Feb. 21, the patient received 0.9 ml of the composition of Example 1 into a 3.89 ml neck lymph node lesion, then experienced a first RT fraction of 7 Gy at day 10 after the injection. A further four fractions of 7 Gy were delivered between days 13-20. An anti-PD1 inhibitor (5×200 mg OD Pembrolizumab followed by 2×400 mg OD

Pembrolizumab) was administered by IV route on day 21 and the treatment is still ongoing. The patient was assessed on 4 May 21, at FUP1 visit, and the response was recorded as partial response (PR) according to RECIST 1.1 and on the next assessment at FUP2 on 15 Jun. 21 the response was assessed as complete response (CR) for target lesions and PR for the overall disease. The patient did not experience any severe adverse effect or dose-limiting toxicity.

1-15. (canceled)

16. A method of treating a solid tumor cancer in a human patient who has had a previous anti-cancer treatment involving radiotherapy (RT) and/or immunotherapy for the treatment of a primary tumor for the same cancer, but who has, at clinical staging:
(i) at least one loco-regional recurrent (LRR) cancerous tumor/lesion in a previously irradiated site, and optionally, 1-5 further metastases, or
(ii) 1-5 metastases, irrespective of the level of control of the previously treated primary tumor,
wherein the nanoparticles and/or aggregates of nanoparticles are selected from hafnium oxide (HfO₂) nanoparticles, rhenium oxide (ReO₂) nanoparticles and any mixture thereof, and the method comprises (a) administering the nanoparticles and/or aggregates of nanoparticles to at least one tumor/lesion or metastasis in the patient, (b) exposing the patient who has been administered with the nanoparticles and/or aggregates of nanoparticles to ionizing radiation and (c) administering at least one immuno-oncology (IO) agent, selected from an anti-PD-1 inhibitor, an anti-PDL-1 inhibitor, an anti-CTLA-4 inhibitor or any mixture thereof, to the patient.

17. The method according to claim 16, wherein the at least one IO agent administered is an anti-PD-1 inhibitor.

18. The method according to claim 16, wherein the nanoparticles and/or aggregates of nanoparticles are administered to only one tumor/lesion or metastasis.

19. The method according to claim 16, wherein the human patient has had a previous anti-cancer treatment involving RT, or RT and immunotherapy and, at clinical staging, has at least one LRR tumor in a previously irradiated site, and, optionally, 1-5 further metastases.

20. The method according to claim 19, wherein the LRR tumor is a head and neck squamous cell carcinoma (HNSCC) LRR tumor, optionally, accompanied by 1-5 metastases.

21. The method according to claim 20, wherein at least one of the metastases is to a lymph node from a HNSCC primary tumor.

22. The method according to claim 16, wherein the previous anti-cancer treatment for the same cancer involved immunotherapy, and wherein said patient, at clinical staging, has 1-5 metastases, irrespective of the level of control of the previously treated primary tumor.

23. The method according to claim 16, wherein the IO agent administered during the previous anti-cancer treatment involving immunotherapy is an anti-PD-1 inhibitor, or an anti-PDL-1 inhibitor, optionally, combined with an anti-CTLA4 antibody.

24. The method according to claim 23, wherein the IO agent administered during the previous anti-cancer treatment involving immunotherapy is an anti-PD-1 inhibitor.

25. The method according to claim 16, wherein the IO agent administered during the previous anti-cancer treatment involving immunotherapy is selected from an anti-PD-1 inhibitor, an anti-PDL-1 inhibitor, an anti-CTLA-4 inhibitor or any mixture thereof, to the patient.

26. The method according to claim 16, wherein the 1-5 metastases are in the lung and/or liver.

27. The method according to claim 16, wherein the patient suffers from solid tumor cancer for whom radiotherapy in combination with immunotherapy using an anti PD-1 inhibitor(s) or anti-PDL-1 inhibitor is indicated.

28. The method according to claim 27, wherein the patient suffers from bladder cancer, metastatic melanoma, (squamous) non-small cell lung cancer (NSCLC), (metastatic) small cell lung cancer (SCLC), (metastatic) head and neck squamous cell cancer (HNSCC), metastatic Urothelial carcinoma, microsatellite Instability (MSI)-high or mismatch repair deficient (dMMR) metastatic solid tumor cancer, colorectal cancer, metastatic gastric cancer, metastatic esophageal cancer, metastatic cervical cancer, or metastatic Merkle cell carcinoma, and wherein the metastases are limited in number to between one and five.

29. The method according to claim 16, wherein the patient is identified as an anti-PD-1 inhibitor non-responder or an anti-PDL1 inhibitor non-responder, and/or for whom monotherapy using an anti-PD-1 inhibitor or an anti-PDL1 inhibitor is not indicated.

30. The method according to claim 16, wherein said nanoparticles and/or aggregates of nanoparticles as are administered in the form of a pharmaceutical composition comprising said nanoparticles and/or aggregates of nanoparticles and a pharmaceutically acceptable carrier or support.
